(19) United States
(12) Patent Application Publication
(10) Pub. No.: US 2025/0229215 A1
(43) Pub. Date: Jul. 17, 2025

(54) CARBON DIOXIDE SEPARATION/CONVERSION DEVICE

(71) Applicant: KYUSHU UNIVERSITY, NATIONAL UNIVERSITY CORPORATION, Fukuoka-shi (JP)

(72) Inventors: Shigenori Fujikawa, Fukuoka-shi (JP); Roman Selyanchyn, Fukuoka-shi (JP); Kenichi Shimizu, Sapporo-shi (JP)

(21) Appl. No.: 18/849,443

(22) PCT Filed: Feb. 7, 2023
(86) PCT No.: PCT/JP2023/004043

§ 371 Date: Sep. 20, 2024

(30) Foreign Application Priority Data
Mar. 23, 2022 (JP) 2022-047646

(51) Int. Cl. B01D 53/14; B01D 53/22; B01D 71/70

(52) U.S. Cl. CPC B01D 53/14; B01D 53/22; B01D 71/70; B01D 2256/20; B01D 2256/245; B01D 2257/504

(57) ABSTRACT

The carbon dioxide separation/conversion device includes: one or more separation units (10) configured to separate carbon dioxide from a carbon dioxide-containing gas to obtain a carbon dioxide-enriched gas; a reaction unit (30) including a catalyst which absorbs and converts the carbon dioxide in the carbon dioxide-enriched gas, and configured to allow the catalyst to absorb the carbon dioxide in the carbon dioxide-enriched gas to obtain a converted gas containing a conversion product formed of the carbon dioxide absorbed by the catalyst and hydrogen; and a hydrogen delivery unit (40) configured to deliver the hydrogen to the reaction unit (30), in which the separation unit (10) includes: one or more separation membrane modules including a separation membrane which separates the carbon dioxide from the carbon dioxide-containing gas; and a separation membrane module connection unit connected to each separation membrane module and including a carbon dioxide delivery port through which the carbon dioxide-enriched gas is discharged.

### TECHNICAL FIELD

The present invention relates to a carbon dioxide separation/conversion device.

Priority is claimed on Japanese Patent Application No. 2022-047646, filed Mar. 23, 2022, the content of which is incorporated herein by reference.

### BACKGROUND ART

Since carbon dioxide (CO₂) discharged from thermal power generation or the like allows global warming, reduction of carbon dioxide is required. In the related art, various technologies have been developed in order to cope with the carbon dioxide discharge problem. Among these, a direct air capture (DAC) technology for directly recovering carbon dioxide in the atmosphere has attracted attention.

As the DAC technology, Patent Document 1 discloses a carbon dioxide capture and treatment system including a carbon dioxide-enriched mixture gas generation device having a separation membrane and increasing a carbon dioxide concentration of a mixture gas taken therein, thereby generating a carbon dioxide-enriched mixture gas; a carbon dioxide conversion device configured to convert carbon dioxide in the enriched mixture gas received from the carbon dioxide-enriched mixture gas generation device, into a chemically stable compound; a final treatment device including an absorbent, in which the final treatment device is configured to absorb the carbon dioxide by the absorbent, thereby separating the carbon dioxide from other gas components; and a carbon dioxide direct capture device configured to take in air contained in an ambient environment, and supply the taken-in air to the final treatment device or an upstream side thereof in the carbon-dioxide capture and treatment system.

### CITATION LIST

### Patent Document

Patent Document 1: PCT International Publication No. WO2021/230045

### SUMMARY OF INVENTION

### Technical Problem

However, when the carbon dioxide is enriched using the separation membrane as in Patent Document 1, oxygen (O₂) is mixed therewith. When a gas containing oxygen and carbon dioxide and hydrogen (H₂) are used as raw materials to generate methane (CH₄) and carbon monoxide (CO) using a catalyst, oxygen preferentially reacts with hydrogen over carbon dioxide, and thus it has been technically difficult to react carbon dioxide with hydrogen.

In addition, in general, in a separation system using a separation membrane, a hollow fiber-type separation membrane module in which hollow fibers are bundled or a spiral-type separation membrane module in which a long separation membrane is formed in a cylindrical shape is used. However, the hollow fiber-type separation membrane module has to be produced in a shape of the hollow fiber, resulting in many production constraints. In addition, the spiral-type separation membrane module uses a large area of the separation membrane at one time, the membrane has to maintain no defects in the large area. Therefore, the system using the separation membrane in the related art has a problem that it is difficult to expand the separation membrane module.

The present invention has been made in view of the above circumstances, and an object of the present invention is to provide a carbon dioxide separation/conversion device that can convert carbon dioxide even if oxygen is contained because of excellent expandability of a separation membrane module.

### Solution to Problem

In order to solve the above problems, the present invention proposes the following aspects.

(1) According to one aspect of the present invention, a carbon dioxide separation/conversion device includes: one or more separation units configured to separate carbon dioxide from a carbon dioxide-containing gas to obtain a carbon dioxide-enriched gas; a reaction unit including a catalyst which absorbs and converts the carbon dioxide in the carbon dioxide-enriched gas, and configured to allow the catalyst to absorb the carbon dioxide in the carbon dioxide-enriched gas to obtain a converted gas containing a conversion product formed of the carbon dioxide absorbed by the catalyst and hydrogen; and a hydrogen delivery unit configured to deliver the hydrogen to the reaction unit, in which the separation unit includes: one or more separation membrane modules including a separation membrane which separates the carbon dioxide from the carbon dioxide-containing gas; a separation membrane module connection unit connected to each separation membrane module and including a carbon dioxide delivery port through which the carbon dioxide-enriched gas is discharged; and a pressure difference formation unit configured to form a pressure difference between an outside of each separation membrane module and the separation membrane module connection unit and an inside of each separation membrane module and the separation membrane module connection unit, the separation membrane module includes: a container including an opening portion and a discharge port through which the carbon dioxide-enriched gas is discharged to the separation membrane module connection unit; and a separation membrane configured to cover the opening portion, is fixed along a circumference of the opening portion, and separates the carbon dioxide from the carbon dioxide-containing gas, the reaction unit includes: one or more reactors having the catalyst; an absorbed gas discharge port configured to discharge an absorbed gas, which is a gas after the catalyst absorbs the carbon dioxide in the carbon dioxide-enriched gas, in each reactor; a converted gas delivery port configured to deliver the converted gas a first gas switching unit connected directly or indirectly to the hydrogen delivery unit, each reactor, and the carbon dioxide delivery port; and a second gas switching unit connected directly or indirectly to the absorbed gas discharge port, each reactor, and the converted gas delivery port, when the catalyst absorbs the carbon dioxide, the first gas switching unit connects at least one of the reactors to the carbon dioxide delivery port, and the second gas switching unit connects the reactor connected to the carbon dioxide delivery port to the absorbed gas discharge port, and when the carbon dioxide absorbed by the catalyst reacts with the hydrogen, the first gas switching unit connects at least one of the reactors to the hydrogen delivery unit, and the second gas switching unit connects the reactor connected to the hydrogen delivery unit to the converted gas delivery port.

(2) In the carbon dioxide separation/conversion device according to (1), the reaction unit may include a first reactor and a second reactor, when the first gas switching unit connects the first reactor to the carbon dioxide delivery port, the first gas switching unit may connect the second reactor to the hydrogen delivery unit in a state where the second reactor and the carbon dioxide delivery port are not connected to each other, and the second gas switching unit may connect the first reactor to the absorbed gas discharge port, and connect the second reactor to the converted gas delivery port in a state where the second reactor and the absorbed gas discharge port are not connected to each other, and when the first gas switching unit may connect the second reactor to the carbon dioxide delivery port, the first gas switching unit may connect the first reactor to the hydrogen delivery unit in a state where the first reactor and the carbon dioxide delivery port are not connected to each other, and the second gas switching unit may connect the second reactor to the absorbed gas discharge port, and connect the first reactor to the converted gas delivery port in a state where the first reactor and the absorbed gas discharge port are not connected to each other.

(3) In the carbon dioxide separation/conversion device according to (1), the reaction unit may include a rust reactor, when the catalyst absorbs the carbon dioxide, the first gas switching unit may connect the first reactor to the carbon dioxide delivery port in a state of not being connected to the hydrogen delivery unit, and the second gas switching unit may connect the first reactor to the absorbed gas discharge port in a state of not being connected to the converted gas delivery port, and when the carbon dioxide absorbed by the catalyst reacts with the hydrogen, the first gas switching unit may connect the first reactor to the hydrogen delivery unit in a state of not being connected to the carbon dioxide delivery port, and the second gas switching unit may connect the first reactor to the converted gas delivery port in a state of not being connected to the absorbed gas discharge port.

(4) In the carbon dioxide separation/conversion device according to any one of (1) to (3), the carbon dioxide-enriched gas discharged from the first separation unit among the separation units may be injected into the second separation unit among the separation units.

(5) In the carbon dioxide separation/conversion device according to any one of (1) to (4), the separation membrane may contain a polydimethylsiloxane-based material as a main component.

(6) The carbon dioxide separation/conversion device according to any one of (1) to (5) may further include: an air blowing unit configured to deliver the carbon dioxide-containing gas to each separation membrane module.

(7) The carbon dioxide separation/conversion device according to any one of (1) to (6) may further include: a moisture removal unit configured to remove moisture in the carbon dioxide-enriched gas.

(8) The carbon dioxide separation/conversion device according to any one of (1) to (7) may further include: a re-injection unit configured to inject the converted gas into each separation membrane module again.

(9) In the carbon dioxide separation/conversion device according to any one of (1) to (8), the conversion product may be carbon monoxide.

(10) In the carbon dioxide separation/conversion device according to any one of (1) to (8), the conversion product may be methane.

### Advantageous Effects of Invention

According to the aspects of the present invention, it is possible to provide a carbon dioxide separation/conversion device that can convert carbon dioxide even if oxygen is contained because of excellent expandability of a separation membrane module.

### BRIEF DESCRIPTION OF DRAWINGS

FIG. 1 is a schematic view of a carbon dioxide separation/conversion device according to a first embodiment.

FIG. 2 is a schematic view of a separation unit of the carbon dioxide separation/conversion device shown in FIG. 1.

FIG. 3 is a plan view of the separation membrane module.

FIG. 4 is a cross-sectional view taken along line A-A of the separation membrane module of FIG. 3.

FIG. 5 is a schematic view of a reaction unit of the carbon dioxide separation/conversion device shown in FIG. 1.

FIG. 6 is a flowchart of a carbon dioxide conversion method according to the first embodiment.

FIG. 7 is a schematic view of a carbon dioxide separation/conversion device according to a second embodiment of the present invention.

FIG. 8 is a schematic view of a reaction unit of the carbon dioxide separation/conversion device shown in FIG. 7.

FIG. 9 is a flowchart of a carbon dioxide conversion method according to the second embodiment.

FIG. 10 is a schematic view of a carbon dioxide separation/conversion device according to a third embodiment.

FIG. 11 is a schematic view of a carbon dioxide separation/conversion device according to a fourth embodiment.

FIG. 12 is a schematic view of a carbon dioxide separation/conversion device according to a fifth embodiment.

FIG. 13 is a graph showing a relationship between a concentration of carbon dioxide, relative humidity, temperature, and time in a carbon dioxide enrichment property test.

FIG. 14 is a graph showing a relationship between a concentration of carbon dioxide, relative humidity, temperature, and time in a carbon dioxide enrichment property test in which moisture removal has been performed.

FIG. 15 is a graph showing a relationship between a concentration of carbon dioxide, relative humidity, temperature, and time in a carbon dioxide enrichment property test in which air has been blown to a separation membrane.

FIG. 16 is a graph showing a relationship between methane generated by a conversion reaction of carbon dioxide and time.

FIG. 17 is a graph showing a relationship between carbon monoxide generated by a conversion reaction of carbon dioxide and time.

### DESCRIPTION OF EMBODIMENTS

### First Embodiment

Hereinafter, a carbon dioxide separation/conversion device according to a first embodiment will be described with reference to the drawings. The drawing used in the following description may be enlarged for convenience in order to make the features of the present invention easy to understand, and the dimensional ratios and the like of each component may differ from the actual ones. Materials, dimensions, and the like provided in the following description are exemplary examples, and the present invention is not limited thereto, and can be appropriately modified and implemented within the range in which effects of the present invention are exhibited.

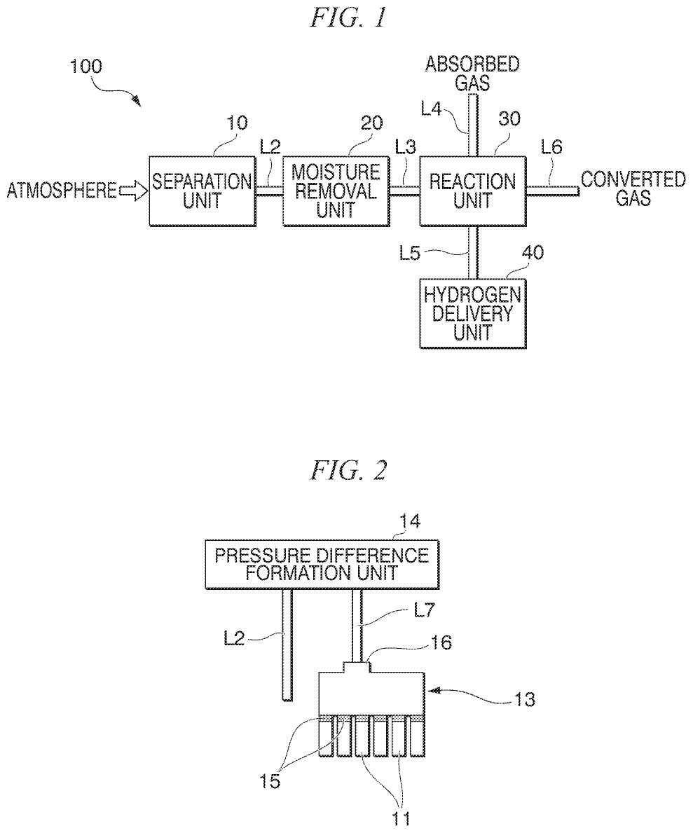

FIG. 1 is a schematic view of a carbon dioxide separation/conversion device 100 according to the first embodiment. A carbon dioxide separation/conversion device 100 includes one or more separation units 10 which separate carbon dioxide from a carbon dioxide-containing gas to obtain a carbon dioxide-enriched gas, a reaction unit 30 which includes a catalyst for absorbing and converting (reducing) the carbon dioxide in the carbon dioxide-enriched gas and causing the catalyst to absorb the carbon dioxide in the carbon dioxide-enriched gas to obtain a converted gas containing a conversion product formed of the carbon dioxide absorbed by the catalyst and hydrogen, and a hydrogen delivery unit 40 which delivers hydrogen to the reaction unit 30. The carbon dioxide separation/conversion device 100 according to the first embodiment can further include a moisture removal unit 20 which removes moisture from the carbon dioxide-enriched gas as necessary. When the moisture removal unit 20 is connected, the separation unit 10 and the moisture removal unit 20 are connected to each other through a flow path L2. The moisture removal unit 20 and the reaction unit 30 are connected to each other through a flow path 13. When the moisture removal unit 20 is not used, the separation unit 10 and the reaction unit 30 are connected to each other through the flow path L2. The reaction unit 30 and the hydrogen delivery unit 40 are connected to each other through a flow path L5. The converted gas generated in the reaction unit 30 passes through a flow path L6 and is delivered to a recovery unit (not shown). The absorbed gas is discharged through a flow path IA. Hereinafter, each unit will be described.

### (Separation Unit 10)

FIG. 2 is a schematic view of the separation unit 10. The separation unit 10 includes one or more separation membrane modules 11 including a separation membrane which separates carbon dioxide from the carbon dioxide-containing gas, a separation membrane module connection unit 13 connected to each separation membrane module 11 and including a carbon dioxide delivery port 16 through which the carbon dioxide-enriched gas is discharged, and a pressure difference formation unit 14 which forms a pressure difference between an outside of each separation membrane module 11 and the separation membrane module connection unit 13 and an inside of each separation membrane module 11 and the separation membrane module connection unit 13. In the first embodiment, the carbon dioxide delivery port 16 is connected to the pressure difference formation unit 14 through the flow path L7, and the pressure difference formation unit 14 is connected to the flow path L2. The carbon dioxide-enriched gas, which has entered from the separation membrane module 11, passes through the carbon dioxide delivery port 16, the flow path L7, and the pressure difference formation unit 14, and is delivered to the reaction unit 30. The present invention is not limited to this configuration as long as the carbon dioxide can be separated from the atmosphere by using the separation membrane module 11 and the carbon dioxide-enriched gas can be delivered to the reaction unit 30.

### “Separation Membrane Module 11”

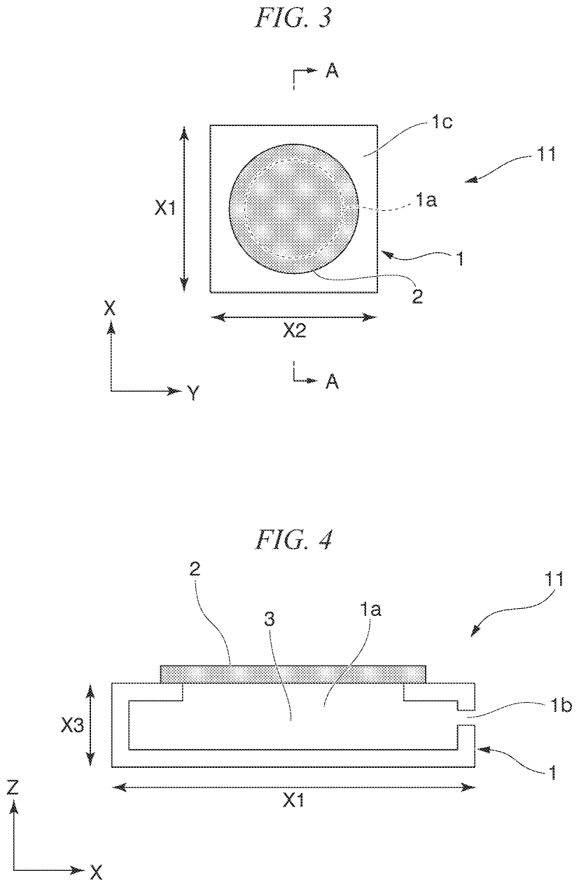

FIG. 3 is a plan view of the separation membrane module 11. FIG. 4 is a cross-sectional view taken along line A-A of the separation membrane module 11 of FIG. 3. In FIGS. 3 and 4, the X direction and the Y direction are directions along a surface 1c of a container 1. The Y direction is perpendicular to the X direction. The Z direction is orthogonal to the X direction and the Y direction. The separation membrane module 11 includes the container 1 including an opening portion 1a and a discharge port 1b through which the carbon dioxide-enriched gas is discharged to the separation membrane module connection unit 13. In addition, the separation membrane module 11 includes a separation membrane 2 which covers the opening portion 1a, is fixed along a circumference of the opening portion 1a, and separates carbon dioxide from the carbon dioxide-containing gas. The carbon dioxide separation/conversion device 100 can easily expand the separation membrane by increasing the number of separation membrane modules 11. The number of separation membrane modules 11 is preferably 2 or more. The separation membrane module 11 may include a pressure sensor (not shown) or the like to monitor the presence or absence of leakage.

The container 1 includes the opening portion 1a, a cavity portion 3, and the discharge port 1b. The carbon dioxide, which has passed through the separation membrane 2 and the opening portion 1a, passes through the cavity portion 3 of the container 1 and is delivered to the separation membrane module connection unit 13 from the discharge port 1b. The opening portion 1a may have a porous support material such as a mesh in order to support the separation membrane 2.

A size of the opening portion 1a is not particularly limited.

The shape of the container 1 is not particularly limited as long as carbon dioxide can permeate the separation membrane 2 and pass through the carbon dioxide delivery port 16 from the discharge port 1b. The shape of the container 1 is preferably a flat plate shape since the number of the separation membrane modules 11 that can be installed in parallel can be increased.

A length X1 of the container 1 in the X direction and a length X2 of the container 1 in the Y direction are not particularly limited. The longer the length X1 and the length X2 of the container 1, the larger the opening portion 1a can have.

A thickness X3 of the container 1 is not particularly limited. Since a large number of separation membrane modules 11 can be connected in parallel to the separation membrane module connection unit 13 as the thickness of the container 1 is thinner, it is preferable that the thickness X3 of the container 1 is thin. The thickness of the container 1 is, for example, 0.1 mm to 10 mm.

The separation membrane 2 has a function of preferentially allowing carbon dioxide in the atmosphere to permeate. Since the separation membrane 2 preferentially allows carbon dioxide in the atmosphere to permeate, the concentration of carbon dioxide in the gas that has passed through the separation membrane 2 is higher than that in the atmosphere. The gas, which has passed through the separation membrane 2 and has an increased concentration of carbon dioxide, is a carbon dioxide-enriched gas.

The material of the separation membrane 2 is not particularly limited as long as it can preferentially allow carbon dioxide in the atmosphere to permeate and can suppress the permeation of components other than carbon dioxide in the atmosphere. Examples of the material of the separation membrane 2 include zeolite, amorphous silica, silica doped with a metal, metal organic frameworks (MOF), carbon, polyimide, polyamide, and a polydimethylsiloxane-based material. In particular, preferably, a separation membrane containing a polydimethylsiloxane-based material as a main component is preferable as the separation membrane 2. The expression “contains polydimethylsiloxane-based material as a main component” means that the amount of the polydimethylsiloxane-based material is 60% by mass or more with respect to the total mass of the separation membrane 2.

The separation membrane 2 covers the opening portion 1a of the container 1 and is fixed along the circumference of the opening portion 1a. The fixing method is not particularly limited as long as atmosphere does not pass through the separation membrane 2 and does not directly enter the container 1 when the pressure difference is formed. For example, the separation membrane 2 may be fixed to the container 1 with an adhesive tape, or the separation membrane 2 may be fixed to the container 1 with an adhesive.

The thickness of the separation membrane 2 is not particularly limited. Since the thinner the thickness of the separation membrane 2, the higher the gas permeability, it is preferable that the separation membrane 2 is thin. The thickness of the separation membrane 2 is, for example, 10 nm to 1 μm.

### “Separation Membrane Module Connection Unit 13”

The separation membrane module connection unit 13 includes a connection unit 15 connected to the separation membrane module 11, and a carbon dioxide delivery port 16. The separation membrane module connection unit 13 is connected to one or more separation membrane modules 11. In the first embodiment, the separation membrane module connection unit 13 is connected to the separation membrane module 11 through the connection unit 15. In addition, the carbon dioxide delivery port 16 of the separation membrane module connection unit 13 is connected to a flow path L7. The separation membrane module connection unit 13 includes a flow path (not shown) therein. The carbon dioxide-enriched gas passes through the discharge port 1b and the connection unit 15 and is discharged from the carbon dioxide delivery port 16.

It is preferable that the connection unit 15 is attachable and detachable to and from the separation membrane module 11. Since the separation membrane module 11 is attachable and detachable by the connection unit 15, only the damaged separation membrane module 11 may be replaced when the separation membrane module 11 is damaged during the conversion of carbon dioxide due to the separation membrane 2 being perforated or the like. Therefore, the attachable and detachable connection unit 15 is provided, so that maintainability can be improved.

### “Pressure Difference Formation Unit 14”

The pressure difference formation unit 14 forms a pressure difference between an outside of each separation membrane module 11 and the separation membrane module connection unit 13 and an inside of each separation membrane module 11 and the separation membrane module connection unit 13. Since there is a pressure difference between the outside of each separation membrane module 11 and the separation membrane module connection unit 13 and the inside of each separation membrane module 11 and the separation membrane module connection unit 13, carbon dioxide in the atmosphere passes through the separation membrane 2 and enters each separation membrane module 11. A pressure on the outside of each separation membrane module 11 and the separation membrane module connection unit 13 may be increased, or a pressure on the inside of each separation membrane module 11 and the separation membrane module connection unit 13 may be decreased.

The pressure difference formation unit 14 is not particularly limited as long as a pressure difference can be formed between the outside of each separation membrane module 11 and the separation membrane module connection unit 13 and the inside of each separation membrane module 11 and the separation membrane module connection unit 13. The pressure difference formation unit 14 is, for example, a diaphragm pump, a dry pump, or a compressor.

### (Reaction Unit)

FIG. 5 is a schematic view of the reaction unit 30. The reaction unit 30 includes one or more reactors 31 having a catalyst 32, an absorbed gas discharge port 33 which discharges an absorbed gas, which is a gas after being absorbed by the catalyst 32, a converted gas delivery port 35 which delivers a converted gas, a first gas switching unit 37 which is connected directly or indirectly to the hydrogen delivery unit 40, each reactor 31, and the carbon dioxide delivery port 16, and a second gas switching unit 39 which is connected directly or indirectly to the absorbed gas discharge port 33, each reactor 31, and the converted gas delivery port 35.

When the catalyst 32 absorbs carbon dioxide, the first gas switching unit 37 connects at least one of the reactors 31 to the carbon dioxide delivery port 16, and the second gas switching unit 39 connects the reactor 31, which is connected to the carbon dioxide delivery port 16, to the absorbed gas discharge port 33. In the first embodiment, when the catalyst 32 absorbs carbon dioxide, the first gas switching unit 37 connects a first reactor 31A to the carbon dioxide delivery port 16 in a state of not being connected to the hydrogen delivery unit 40, and the second gas switching unit 39 connects the first reactor 31A to the absorbed gas discharge port 33 in a state of not being connected to the converted gas delivery port 35. In this way, the reactor 31 or the like is connected to allow the carbon dioxide-enriched gas to flow into the reactor 31, so that the catalyst 32 can absorb carbon dioxide. The absorbed gas after carbon dioxide is absorbed passes through the absorbed gas discharge port 33 and is discharged from the flow path L4. The absorbed gas contains oxygen and the like. Accordingly, the absorbed gas is discharged, so that components other than carbon dioxide can be removed.

When carbon dioxide absorbed by the catalyst 32 reacts with hydrogen, the first gas switching unit 37 connects at least one of the reactors 31 to the hydrogen delivery unit 40, and the second gas switching unit 39 connects the reactor 31, which is connected to the hydrogen delivery unit 40, to the converted gas delivery port 35.

In the first embodiment, when carbon dioxide absorbed by the catalyst 32 reacts with hydrogen, the first gas switching unit 37 connects the first reactor 31A to the hydrogen delivery unit 40 in a state of not being connected to the carbon dioxide delivery port 16, and the second gas switching unit 39 connects the first reactor 31A to the converted gas delivery port 35 in a state of not being connected to the absorbed gas discharge port 33. In this way, the reactor 31 or the like is to allow hydrogen to flow into the reactor 31 in which the catalyst 32 absorbs carbon dioxide, so that carbon dioxide can be converted, thereby obtaining a conversion product. The converted gas containing a conversion product obtained by converting carbon dioxide passes through the converted gas delivery port 35 and is delivered from the flow path L6. The delivered converted gas is delivered to a recovery unit (not shown).

### “Reactor”

The reactor 31 includes a storage container 34 and a catalyst 32 in the storage container 34. In the first embodiment, the number of reactors 31 is one. That is, in the first embodiment, the reactor 31 is only the first reactor 31A, but the present invention is not limited thereto. In the present invention, a plurality of reactors 31 may be used. The first reactor 31A is connected to the first gas switching unit 37 through a flow path L8. The first reactor 31A is connected to the second gas switching unit 39 through a flow path L9. When the carbon dioxide-enriched gas flowing from the first gas switching unit 37 passes through the first reactor 31A, the carbon dioxide-enriched gas comes into contact with the catalyst 32, and carbon dioxide is absorbed. After carbon dioxide is absorbed by the catalyst 32, carbon dioxide is converted using hydrogen delivered from the hydrogen delivery unit 40 to generate a conversion product.

The catalyst 32 is not particularly limited as long as it can absorb and convert carbon dioxide. The catalyst 32 can be selected according to a target conversion product. The catalyst 32 includes, for example, a carrier, and a basic substance and a transition metal which are supported on the carrier.

The carrier of the catalyst 32 is not particularly limited as long as it can support the basic substance and the transition metal. More specifically, the carrier of the catalyst 32 is preferably a material that does not participate in absorption and conversion reaction of carbon dioxide, and is preferably a material having durability required as a carrier. The shape thereof is preferably porous in order to enhance the reaction effect. The material of the carrier is preferably a metal oxide, and more specifically, for example, alumina (Al₂O₃), silica (SiO₂), magnesia (MgO), zirconia (ZrO₂), titania (TiO₂), ceria (CeO₂), and the like. Among these, in consideration of characteristics as a catalyst, alumina is particularly preferable as the carrier.

The basic substance is, for example, an alkali metal or an alkaline earth metal. The basic substance contributes to absorption of carbon dioxide and generation of the conversion product. The basic substance is sodium (Na), potassium (K), or calcium (Ca). The basic substance reacts with carbon dioxide to form a carbonate. That is, the catalyst 32 absorbs carbon dioxide by forming a reaction product with carbon dioxide. The basic substance is appropriately selected according to the target conversion product and the transition metal supported by the carrier. When methane is generated from carbon dioxide, calcium is preferable as the basic substance. When carbon monoxide is generated from carbon dioxide, sodium is preferable as the basic substance.

The transition metal contributes to the generation of the conversion product. Examples of the transition metal include platinum (Pt), nickel (Ni), and ruthenium (Ru). When methane is generated from carbon dioxide, Ni is preferable as the transition metal. When carbon monoxide is generated from carbon dioxide, Pt is preferable as the transition metal.

For example, when carbon dioxide is converted to generate methane, the catalyst 32 is preferably a catalyst in which Ni nanoparticles and calcium are carried on alumina. When carbon dioxide is converted to generate carbon monoxide, the catalyst 32 is preferably a catalyst in which Pt nanoparticles and sodium are supported on alumina.

The transition metal is preferably present in a particulate form. In addition, a particle diameter of particles formed of the transition metal is, for example, 1 to 30 nm. The particle diameter of the particles can be confirmed from an observation image obtained by observation with a scanning transmission electron microscope (STEM).

### “First Gas Switching Unit 37”

The first gas switching unit 37 according to the first embodiment is connected to the carbon dioxide delivery port 16 through the flow path L3. The first gas switching unit 37 is connected to the hydrogen delivery unit 40 through the flow path L5. The first gas switching unit 37 is connected to the first reactor 31A through the flow path L8. The first gas switching unit 37 is, for example, a three-way valve.

### “Second Gas Switching Unit 39”

The second gas switching unit 39 according to the first embodiment is connected to the first reactor 31A through the flow path L9. The second gas switching unit 39 includes the absorbed gas discharge port 33 and the converted gas delivery port 35. The absorbed gas discharge port 33 is connected to the flow path L4. The converted gas delivery port 35 is connected to the flow path L6. The second gas switching unit 39 is, for example, a three-way valve.

### (Moisture Removal Unit)

The moisture removal unit 20 removes moisture in the carbon dioxide-enriched gas obtained by the separation unit 10. The moisture removal unit 20 is connected to the separation unit 10 through the flow path L2. In addition, the moisture removal unit 20 is connected to the reaction unit 30 through the flow path L3. In the first embodiment, the carbon dioxide-enriched gas used in the reaction unit 30 is a gas from which moisture has been removed. As the moisture removal method, a known method can be used. Examples of the moisture removal unit 20 include a cooling-type water trap and a molecular sieve unit including a molecular sieve. The moisture removal unit 20 may include, for example, a plurality of moisture removal units 20 such as a cooling-type water trap and a molecular sieve.

### (Hydrogen Delivery Unit)

The hydrogen delivery unit 40 delivers hydrogen to the reaction unit 30. Specifically, the gas is delivered to the first reactor 31A through the flow path L5 and the first gas switching unit 37. The hydrogen delivery unit 40 is not particularly limited as long as it can deliver hydrogen to the reaction unit 30. The hydrogen delivery unit 40 is, for example, a hydrogen cylinder that stores hydrogen.

Hereinabove, the carbon dioxide separation/conversion device 100 according to the first embodiment has been described above. According to the carbon dioxide separation/conversion device 100 according to the first embodiment, it is possible to convert carbon dioxide even when oxygen is contained because of excellent expandability of the separation membrane module 11.

In the first embodiment, the carbon dioxide separation/conversion device 100 includes the moisture removal unit 20, but the moisture removal unit 20 may not be provided.

### <Carbon Dioxide Conversion Method>

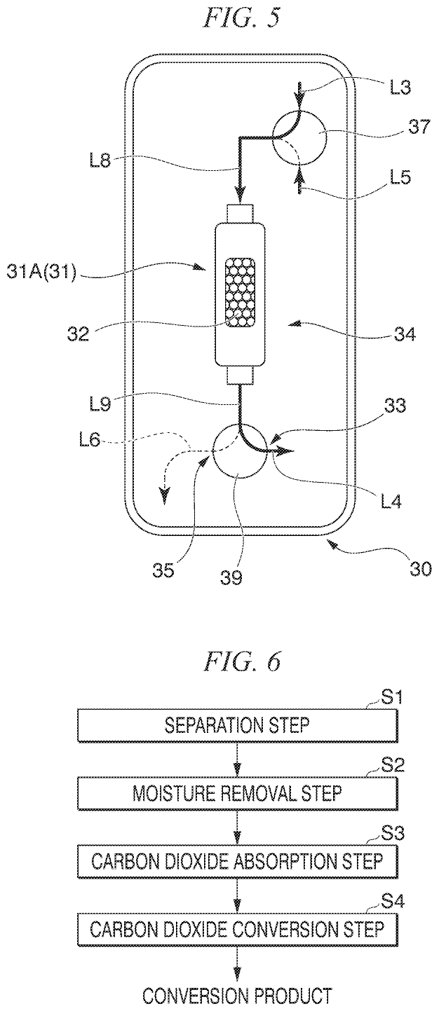

Next, a carbon dioxide conversion method according to the present embodiment will be described. An example in which the carbon dioxide separation/conversion device 100 according to the present embodiment is used will be described, but the carbon dioxide conversion method according to the present embodiment is not limited to the following method. FIG. 6 is a flowchart of the carbon dioxide conversion method according to the first embodiment. The carbon dioxide conversion method of the first embodiment includes a separation step S1 of separating carbon dioxide from the atmosphere and increasing a concentration of carbon dioxide to obtain a carbon dioxide-enriched gas, a moisture removal step S2 of removing moisture from the carbon dioxide-enriched gas, a carbon dioxide absorption step S3 of allowing the catalyst 32 to absorb carbon dioxide in the carbon dioxide-enriched gas, and a carbon dioxide conversion step S4 of obtaining a converted gas containing a conversion product from the carbon dioxide absorbed by the catalyst 32 and hydrogen.

### (Separation Step)

In the separation step S1, carbon dioxide is separated from the atmosphere, and the concentration of carbon dioxide is increased to obtain a carbon dioxide-enriched gas. Specifically, the separation membrane 2 of the separation membrane module 11 is used to separate and enrich carbon dioxide in the atmosphere. The carbon dioxide-enriched gas that has permeated into the separation membrane 2 passes through the inside of the separation membrane module 11 and the separation membrane module connection unit 13, and is delivered from the carbon dioxide delivery port 16 to the moisture removal unit 20.

In order to allow carbon dioxide in the atmosphere to permeate the separation membrane 2, the pressure difference formation unit 14 forms a pressure difference between the outside of each separation membrane module 11 and the separation membrane module connection unit 13 and the inside of each separation membrane module 11 and the separation membrane module connection unit 14.

For example, carbon dioxide may be allowed to permeate from the separation membrane 2 into the inside of each separation membrane module 11 and the separation membrane module connection unit 13 by depressurizing the inside of each separation membrane module 11 and the separation membrane module connection unit 13 to 10 kPa or less using a diaphragm pump.

### (Moisture Removal Step)

In the moisture removal step S2, moisture is removed from the carbon dioxide-enriched gas. Specifically, moisture is removed from the carbon dioxide-enriched gas, which has passed through the flow path L2 and entered the moisture removal unit 20, and the carbon dioxide-enriched gas after the moisture is removed is delivered to the reaction unit 30 through the flow path L3. In the moisture removal step S2, for example, moisture in the carbon dioxide-enriched gas may be removed using a cooling-type water trap and a molecular sieve.

### (Carbon Dioxide Absorption Step)

In the carbon dioxide absorption step S3, the catalyst 32 absorbs carbon dioxide in the carbon dioxide-enriched gas. Specifically, the carbon dioxide-enriched gas after the moisture is removed by the moisture removal unit 20 is allowed to flow into the first reactor 31A. As a result, carbon dioxide in the carbon dioxide-enriched gas comes into contact with the catalyst 32 and is absorbed by the catalyst 32. The absorbed gas after the catalyst 32 is absorbed carbon dioxide passes through the flow path L9 from the first reactor 31A and is discharged from the absorbed gas discharge port 33. The absorbed gas contains unnecessary components such as oxygen. The absorbed gas is discharged, so that components other than carbon dioxide, such as oxygen, are removed. The absorbed gas after being discharged from the absorbed gas discharge port 33 passes through the flow path L4 and is discharged into, for example, the atmosphere.

In the carbon dioxide absorption step S3, the first gas switching unit 37 connects at least one of the reactors 31 to the carbon dioxide delivery port 16, and the second gas switching unit 39 connects the reactor 31, which is connected to the carbon dioxide delivery port 16, to the absorbed gas discharge port 33. In the carbon dioxide absorption step S3 of the first embodiment, the first gas switching unit 37 connects the first reactor 31A to the carbon dioxide delivery port 16 in a state of not being connected to the hydrogen delivery unit 40, and the second gas switching unit 39 connects the first reactor 31A to the absorbed gas discharge port 33 in a state of not being connected to the converted gas delivery port 35.

In the carbon dioxide absorption step S3, it is preferable to heat the first reactor 31A. By heating, a subsequent carbon dioxide conversion reaction can be quickly performed. A temperature of the first reactor 31A can be appropriately set according to a type of the catalyst used and a type of the conversion product. It is preferable to maintain the temperature of the first reactor 31A at 300° C. to 450° C.

### (Carbon Dioxide Conversion Step)

In the carbon dioxide conversion step S4, a converted gas containing a conversion product is obtained from carbon dioxide absorbed by the catalyst 32 and hydrogen. Specifically, hydrogen delivered from the hydrogen delivery unit 40 is allowed to flow into the first reactor 31A after the carbon dioxide absorption step S3 is completed. On the catalyst 32 in the first reactor 31A, carbon dioxide is converted (reduced) from the adsorbed carbon dioxide and hydrogen, thereby generating a conversion product. The conversion product is, for example, methane or carbon monoxide. The converted gas containing the conversion product after the conversion reaction passes through the flow path L9 and is delivered from the converted gas delivery port 35 of the second gas switching unit 39. The converted gas is transported to, for example, a recovery unit (not shown), but the converted gas may be injected into another system as it is.

In the carbon dioxide conversion step S4, the first gas switching unit 37 connects at least one of the reactors 31 to the hydrogen delivery unit 40, and the second gas switching unit 39 connects the reactor 31, which is connected to the hydrogen delivery unit 40, to the converted gas delivery port 35. In the carbon dioxide conversion step S4 of the first embodiment, when carbon dioxide absorbed by the catalyst 32 reacts with hydrogen, the first gas switching unit 37 connects the first reactor 31A to the hydrogen delivery unit 40 in a state of not being connected to the carbon dioxide delivery port 16, and the second gas switching unit 39 connects the first reactor 31A to the converted gas delivery port 35 in a state of not being connected to the absorbed gas discharge port 33. In this way, the reactor 31 or the like is to allow hydrogen to flow into the reactor 31 in which the catalyst 32 absorbs carbon dioxide, so that carbon dioxide can be converted, thereby obtaining a conversion product.

As the catalyst 32, the above-described catalyst can be used. For example, when carbon dioxide is converted to generate methane, the catalyst 32 is preferably a catalyst in which Ni nanoparticles and calcium are carried on alumina. When carbon dioxide is converted to generate carbon monoxide, the catalyst 32 is preferably a catalyst in which Pt nanoparticles and sodium are supported on alumina.

In the carbon dioxide conversion step S4, it is preferable to heat the first reactor 31A. By heating, the conversion reaction of carbon dioxide can be promoted. A temperature of the first reactor 31A can be appropriately set according to a type of the catalyst used and a type of the conversion product. It is preferable to maintain the temperature of the first reactor 31A at 300° C. to 450° C.

Hereinabove, the carbon dioxide conversion method according to the first embodiment has been described above. According to the carbon dioxide conversion method according to the first embodiment, it is possible to convert carbon dioxide even when oxygen is contained because of excellent expandability of the separation membrane module 11.

### (Method for Producing Catalyst)

The catalyst can be produced by a known method. The method for producing the catalyst is, for example, a wet-type impregnation method. An aqueous solution of a compound formed of a basic substance is impregnated into a carrier (for example, alumina), dried, and sintered. Next, a solid after the sintering is impregnated with an aqueous solution of a transition metal compound, dried, and sintered. As a result, a catalyst 32 is obtained.

### Second Embodiment

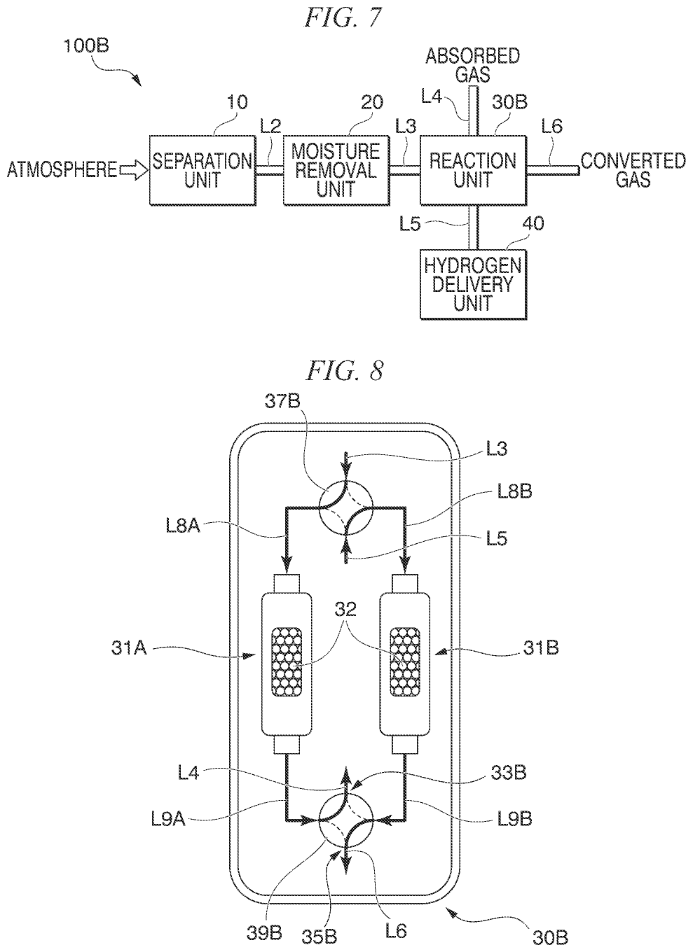

Next, a carbon dioxide separation/conversion device 100B according to a second embodiment of the present invention will be described with reference to FIG. 7. In addition, in the second embodiment, the same portions as the constituent elements in the above-described first embodiment will be designated by the same reference signs, and a description thereof will be omitted, and only different points will be described.

FIG. 7 is a schematic view of a carbon dioxide separation/conversion device 100B according to a second embodiment. The carbon dioxide separation/conversion device 100B includes one or more separation units 10 which separate carbon dioxide from a carbon dioxide-containing gas to obtain a carbon dioxide-enriched gas, a reaction unit 30B which includes a catalyst for absorbing and converting the carbon dioxide in the carbon dioxide-enriched gas and causing the catalyst to absorb the carbon dioxide in the carbon dioxide-enriched gas to obtain a converted gas containing a conversion product formed of the carbon dioxide absorbed by the catalyst and hydrogen, and a hydrogen delivery unit 40 which delivers hydrogen to the reaction unit 30B. The carbon dioxide separation/conversion device 100B according to the second embodiment further includes a moisture removal unit 20 which removes moisture from the carbon dioxide-enriched gas. The separation unit 10 and the moisture removal unit 20 are connected to each other through a flow path L2. The moisture removal unit 20 and the reaction unit 30B are connected to each other through a flow path L3. The reaction unit 30B and the hydrogen delivery unit 40 are connected to each other through a flow path L5. The converted gas generated in the reaction unit 30B passes through the flow path L6 and is delivered to a recovery unit (not shown). The absorbed gas is discharged through a flow path IA. Hereinafter, each unit will be described.

### (Reaction Unit)

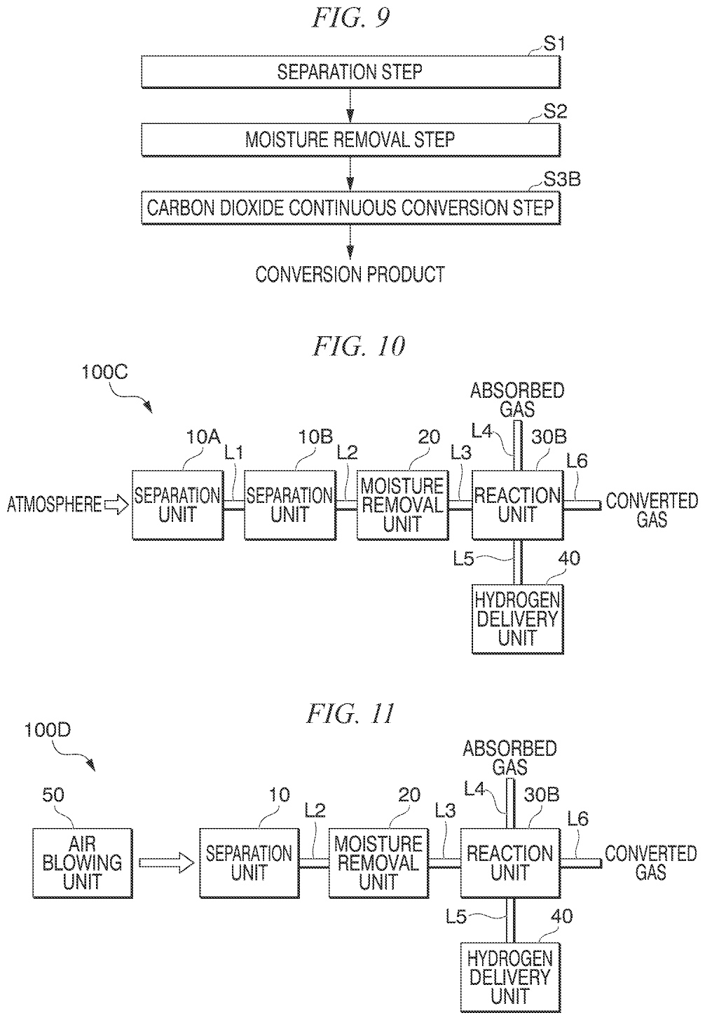

FIG. 8 is a schematic view of the reaction unit 30B. The reaction unit 30B includes one or more reactors 31 having a catalyst 32, an absorbed gas discharge port 33B which discharges an absorbed gas, which is a gas after the catalyst 32 absorbs carbon dioxide in the carbon dioxide-enriched gas, a converted gas delivery port 35B which delivers a converted gas, a first gas switching unit 37B which is connected directly or indirectly to the hydrogen delivery unit 40, each reactor 31, and the carbon dioxide delivery port 16, and a second gas switching unit 39B which is connected directly or indirectly to the absorbed gas discharge port 33B, each reactor 31, and the converted gas delivery port 35B. The reaction unit 30B of the carbon dioxide separation/conversion device 100B according to the second embodiment includes a first reactor 31A and a second reactor 31B.

When the first gas switching unit 37B connects the first reactor 31A to the carbon dioxide delivery port 16, the first gas switching unit 37B connects the second reactor 31B to the hydrogen delivery unit 40 in a state of not being connected to the second reactor 31B and the carbon dioxide delivery port 16, and the second gas switching unit 39B connects the first reactor 31A to the absorbed gas discharge port 33B and connects the second reactor 31B to the converted gas delivery port 35B in a state of not being connected to the second reactor 31B and the absorbed gas discharge port 33B. In this way, the reactors are connected to allow carbon dioxide-enriched gas to flow into the first reactor 31A and hydrogen to flow into the second reactor 31B, so that absorption of carbon dioxide by the catalyst 32 can be performed in the first reactor 31A, and simultaneously, the conversion reaction of carbon dioxide can be performed in the second reactor 31B. The absorbed gas after absorbing carbon dioxide in the first reactor 31A passes through the absorbed gas discharge port 33B and is discharged from the flow path L4. The absorbed gas contains oxygen and the like. Accordingly, the absorbed gas is discharged, so that components other than carbon dioxide can be removed. The converted gas, which contains a conversion product obtained by converting carbon dioxide in the second reactor 31B, passes through the converted gas delivery port 35B and is delivered from the flow path L6. The delivered converted gas is delivered to a recovery unit (not shown).

When the first gas switching unit 37B connects the second reactor 31B to the carbon dioxide delivery port 16, the first gas switching unit 37B connects the first reactor 31A to the hydrogen delivery unit 40 in a state of not being connected to the first reactor 31A and the carbon dioxide delivery port 16, and the second gas switching unit 39B connects the second reactor 31B to the absorbed gas discharge port 33B and connects the first reactor 31A to the converted gas delivery port 35B in a state of not being connected to the first reactor 31A and the absorbed gas discharge port 33B. The conversion reaction of carbon dioxide can be performed in the first reactor 31A, and simultaneously, absorption of carbon dioxide into the catalyst 32 can be performed in the second reactor 31B. The absorbed gas after absorbing carbon dioxide in the second reactor 31B passes through the absorbed gas discharge port 33B and is discharged from the flow path L4. The absorbed gas contains oxygen and the like. Accordingly, the absorbed gas is discharged, so that components other than carbon dioxide can be removed. The converted gas, which contains a conversion product obtained by converting carbon dioxide in the first reactor 31A, passes through the converted gas delivery port 35B and is delivered from the flow path L6. The delivered converted gas is delivered to a recovery unit (not shown).

### “Reactor”

The reactor 31 includes a storage container 34 and a catalyst 32 in the storage container 34. In the second embodiment, the number of reactors 31 is two. That is, in the second embodiment, the reactor 31 has two reactors, that is, the first reactor 31A and the second reactor 31B, but the present invention is not limited thereto. In the present invention, two or more reactors 31 may be used. The first reactor 31A is connected to the first gas switching unit 37B through a flow path L8A. The first reactor 31A is connected to the second gas switching unit 39 through a flow path L9A. The second reactor 31B is connected to the first gas switching unit 37B through a flow path L8B. The second reactor 31B is connected to the second gas switching unit 39B through a flow path L9B. When the carbon dioxide-enriched gas flowing from the first gas switching unit 37B passes through the first reactor 31A or the second reactor 31B, the carbon dioxide-enriched gas comes into contact with the catalyst 32, and carbon dioxide is absorbed. After carbon dioxide is absorbed by the catalyst 32, carbon dioxide is converted using hydrogen delivered from the hydrogen delivery unit 40 to generate a conversion product. The conversion reaction is, for example, a reduction reaction.

### “First Gas Switching Unit”

The first gas switching unit 37B according to the second embodiment is connected to the carbon dioxide delivery port 16 through the flow path 13. The first gas switching unit 37B is connected to the hydrogen delivery unit 40 through the flow path L5. The first gas switching unit 37B is connected to the first reactor 31A through the flow path L8A. The first gas switching unit 37B is connected to the second reactor 31B through the flow path L8B. The first gas switching unit 37B is, for example, a four-way valve.

### “Second Gas Switching Unit”

The second gas switching unit 39B according to the second embodiment is connected to the first reactor 31A through the flow path L9A. The second gas switching unit 39B is connected to the second reactor 31B through the flow path L9B. The second gas switching unit 39B includes the absorbed gas discharge port 33B and the converted gas delivery port 35B. The absorbed gas discharge port 33B is connected to the flow path L4. The converted gas delivery port 35B is connected to the flow path L6. The second gas switching unit 39B is, for example, a four-way valve.

Hereinabove, the carbon dioxide separation/conversion device 100B according to the second embodiment has been described above. According to the carbon dioxide separation/conversion device 100B according to the second embodiment, it is possible to convert carbon dioxide even when oxygen is contained because of excellent expandability of the separation membrane module 11. In addition, the absorption and conversion reaction of carbon dioxide can be simultaneously performed.

In the second embodiment, the carbon dioxide separation/conversion device 100 includes the moisture removal unit 20, but the moisture removal unit 20 may not be provided.

### <Carbon Dioxide Conversion Method>

Next, a carbon dioxide conversion method according to the second embodiment will be described. An example in which the carbon dioxide separation/conversion device 100B according to the second embodiment is used will be described, but the carbon dioxide conversion method according to the present embodiment is not limited to the following method. FIG. 9 is a flowchart of the carbon dioxide conversion method according to the second embodiment. The carbon dioxide conversion method of the second embodiment includes a separation step S1 of separating carbon dioxide from the atmosphere and increasing a concentration of carbon dioxide to obtain a carbon dioxide-enriched gas, a moisture removal step S2 of removing moisture from the carbon dioxide-enriched gas, and a carbon dioxide continuous conversion step S3B of absorbing carbon dioxide in the carbon dioxide-enriched gas into the catalyst 32 in one reactor 31, and simultaneously, obtaining a converted gas containing a conversion product from carbon dioxide and hydrogen in another reactor 31.

### (Carbon Dioxide Continuous Conversion Step)

In the carbon dioxide continuous conversion step S3B, carbon dioxide is absorbed by the catalyst 32 of one reactor 31 (for example, the first reactor 31A), and simultaneously, a conversion product is generated from carbon dioxide and hydrogen on the catalyst 32 of another reactor 31 (for example, the second reactor 31B). Hereinafter, an example of the first reactor 31A and the second reactor 31B will be described. To absorb carbon dioxide into the catalyst 32 of the first reactor 31A, the first gas switching unit 37B connects the first reactor 31A to the carbon dioxide delivery port 16 to allow a carbon dioxide-enriched gas to flow. Simultaneously, the first gas switching unit 37B connects the hydrogen delivery unit 40 to the second reactor 31B to allow hydrogen to flow in a state where the carbon dioxide delivery port 16 and the second reactor 31B are not connected to each other such that the carbon dioxide-enriched gas does not flow. As a result, in the first reactor 31A, carbon dioxide in the carbon dioxide-enriched gas comes into contact with the catalyst 32 and is absorbed by the catalyst 32. Simultaneously, in the second reactor 31B, a conversion product is generated from carbon dioxide and hydrogen. In one hydrogen flow path L5 and one carbon dioxide-enriched gas flow path L3, by changing the reactor 31 to which each flow path is connected by the first gas switching unit 37B and the second gas switching unit 39B, carbon dioxide can be continuously converted. The absorbed gas after carbon dioxide is absorbed by the catalyst 32 of the first reactor 31A passes through the flow path L9A from the first reactor 31A and is discharged from the absorbed gas discharge port 33B. The absorbed gas contains unnecessary components such as oxygen. The absorbed gas is discharged, so that components other than carbon dioxide, such as oxygen, are removed. The absorbed gas after being discharged from the absorbed gas discharge port 33B passes through the flow path L4 and is discharged into, for example, the atmosphere. The converted gas containing the conversion product passes through the flow path L9B and the flow path L6 from the second reactor 31B and is delivered to the recovery unit.

As the catalyst 32, the above-described catalyst can be used. It is preferable that the catalysts 32 of the first reactor 31A and the second reactor 31B are the same. For example, when carbon dioxide is converted to generate methane, the catalysts 32 of the first reactor 31A and the second reactor 31B are preferably catalysts in which Ni nanoparticles and calcium are supported on alumina. When carbon dioxide is converted to generate carbon monoxide, the catalysts 32 of the first reactor 31A and the second reactor 31B are preferably catalysts in which Pt nanoparticles and sodium are supported on alumina.

In the carbon dioxide continuous conversion step S3B, it is preferable to heat the first reactor 31A and the second reactor 31B. By heating, the adsorption and conversion reaction of carbon dioxide can be promoted. The temperatures of the first reactor 31A and the second reactor 31B can be appropriately set according to the type of the catalyst used and the type of the conversion product. It is preferable that the temperatures of the first reactor 31A and the second reactor 31B are the same. The temperatures of the first reactor 31A and the second reactor 31B are preferably maintained, for example, 300° C. to 450° C.

Hereinabove, the carbon dioxide conversion method according to the second embodiment has been described above. According to the carbon dioxide conversion method according to the second embodiment, it is possible to convert carbon dioxide even when oxygen is contained because of excellent expandability of the separation membrane module 11. In addition, according to the carbon dioxide conversion method according to the second embodiment, carbon dioxide can be continuously converted.

### Third Embodiment

Next, a carbon dioxide separation/conversion device 100C according to a third embodiment of the present invention will be described with reference to FIG. 10. In addition, in the third embodiment, the same portions as the constituent elements in the above-described first and second embodiments will be designated by the same reference signs, and a description thereof will be omitted, and only different points will be described.

FIG. 10 is a schematic view of a carbon dioxide separation/conversion device 100C according to the third embodiment. The carbon dioxide separation/conversion device 100C includes a first separation unit 10A and a second separation unit 10B which separate carbon dioxide from a carbon dioxide-containing gas to obtain a carbon dioxide-enriched gas, a reaction unit 30B which includes a catalyst for absorbing and converting the carbon dioxide in the carbon dioxide-enriched gas and causing the catalyst to absorb the carbon dioxide in the carbon dioxide-enriched gas to obtain a converted gas containing a conversion product formed of the carbon dioxide absorbed by the catalyst and hydrogen, and a hydrogen delivery unit 40 which delivers hydrogen to the reaction unit 30B. The carbon dioxide separation/conversion device 100C according to the third embodiment further includes a moisture removal unit 20 which removes moisture from the carbon dioxide-enriched gas. The first separation unit 10A and the second separation unit 10B are connected to each other through a flow path L1. The second separation unit 10B and the moisture removal unit 20 are connected to each other through a flow path 12. The moisture removal unit 20 and the reaction unit 30B are connected to each other through a flow path L3. The reaction unit 30B and the hydrogen delivery unit 40 are connected to each other through a flow path L5. The converted gas generated in the reaction unit 30B passes through the flow path L6 and is delivered to a recovery unit (not shown). The absorbed gas is discharged through a flow path L4. Hereinafter, each unit will be described.

In the carbon dioxide separation/conversion device 100C, the first separation unit 10A and the second separation unit JOB are connected in series. Here, the expression “connected in series” means the first separation unit 10A and the second separation unit LOB are connected such that the carbon dioxide-enriched gas discharged from one separation unit 10 (for example, the first separation unit 10A) can be injected into the separation membrane module 11 of the other separation unit 10 (for example, the second separation unit 10B). By connecting the first separation unit 10A and the second separation unit 10B in series, carbon dioxide is separated from the carbon dioxide-enriched gas that has been once enriched, so that the carbon dioxide concentration in the carbon dioxide-enriched gas can be further increased.

Hereinabove, the carbon dioxide separation/conversion device 100C according to the third embodiment has been described above. According to the carbon dioxide separation/conversion device 100C according to the third embodiment, it is possible to convert carbon dioxide even when oxygen is contained because of excellent expandability of the separation membrane module 11. In addition, the absorption and conversion reaction of carbon dioxide can be simultaneously performed. Moreover, since the first separation unit 10A and the second separation unit 10B are connected in series, the carbon dioxide concentration in the carbon dioxide-enriched gas can be further increased.

In the third embodiment, in the carbon dioxide separation/conversion device 100C, the first separation unit 10A and the second separation unit 10B are connected in series, but the first separation unit 10A and the second separation unit 10B may be connected in parallel. The expression “connected in parallel” herein means that the first separation unit 10A and the second separation unit 10B are connected such that the gas before the separation of carbon dioxide is the same and the carbon dioxide-enriched gases separated in the first separation unit 10A and the second separation unit 10B are joined to one separation unit. By connecting in parallel, the amount of gas that can be separated at once can be increased.

### Fourth Embodiment

Next, a carbon dioxide separation/conversion device 100D according to a fourth embodiment of the present invention will be described with reference to FIG. 11. In addition, in the fourth embodiment, the same portions as the constituent elements in the above-described first, second, and third embodiments will be designated by the same reference signs, and a description thereof will be omitted, and only different points will be described.

FIG. 11 is a schematic view of the carbon dioxide separation/conversion device 100D according to the fourth embodiment. The carbon dioxide separation/conversion device 100D includes one or more separation units 10 which separate carbon dioxide from a carbon dioxide-containing gas to obtain a carbon dioxide-enriched gas, a reaction unit 30B which includes a catalyst for absorbing and converting the carbon dioxide in the carbon dioxide-enriched gas and causing the catalyst to absorb the carbon dioxide in the carbon dioxide-enriched gas to obtain a converted gas containing a conversion product formed of the carbon dioxide absorbed by the catalyst and hydrogen, and a hydrogen delivery unit 40 which delivers hydrogen to the reaction unit 30B. The carbon dioxide separation/conversion device 100D according to the fourth embodiment further includes a moisture removal unit 20 which removes moisture from the carbon dioxide-enriched gas, and an air blowing unit 50 which delivers a carbon dioxide-containing gas to each separation membrane module 11. The separation unit 10 and the moisture removal unit 20 are connected to each other through a flow path L2. The moisture removal unit 20 and the reaction unit 30B are connected to each other through a flow path L3. The reaction unit 30B and the hydrogen delivery unit 40 are connected to each other through a flow path L5. The converted gas generated in the reaction unit 30B passes through the flow path L6 and is delivered to a recovery unit (not shown). The absorbed gas is discharged through a flow path L4. Hereinafter, each unit will be described.

When the separation unit 10 separates carbon dioxide from the carbon dioxide-containing gas (for example, the atmosphere), the concentration of carbon dioxide in the carbon dioxide-containing gas around the separation membrane 2 decreases. Therefore, when the separation unit 10 continues to separate carbon dioxide from the carbon dioxide-containing gas from a gas (for example, the atmosphere), an increase in the concentration of carbon dioxide in the carbon dioxide-enriched gas is suppressed. The air blowing unit 50 delivers the carbon dioxide-containing gas (for example, the atmosphere) to the separation membrane module 11, so that the concentration of the carbon dioxide around the separation membrane 2 can be maintained constantly. As a result, the concentration of the carbon dioxide-enriched gas obtained by separating carbon dioxide in the separation unit 10 can be further increased.

Hereinabove, the carbon dioxide separation/conversion device 100D according to the fourth embodiment has been described. According to the carbon dioxide separation/conversion device 1001) according to the fourth embodiment, it is possible to convert carbon dioxide even when oxygen is contained because of excellent expandability of the separation membrane module 11. In addition, the absorption and conversion reaction of carbon dioxide can be simultaneously performed. Moreover, the air blowing unit 50 delivers the carbon dioxide-containing gas (for example, the atmosphere), so that the concentration of the carbon dioxide around the separation membrane 2 can be maintained constantly. As a result, the concentration of the carbon dioxide-enriched gas obtained by separating carbon dioxide in the separation unit 10 can be further increased.

### Fifth Embodiment

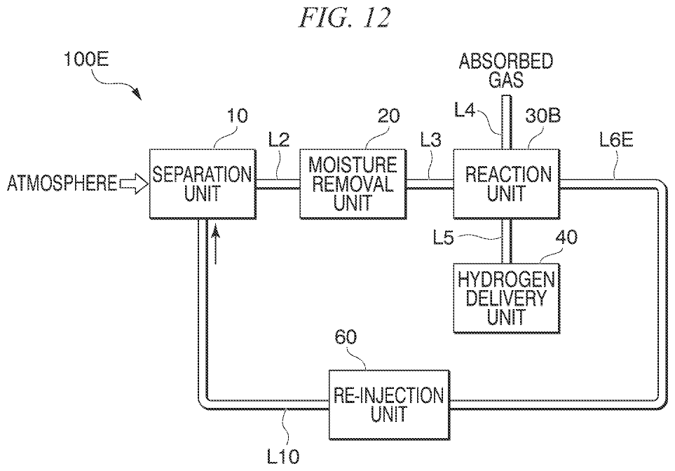

Next, a carbon dioxide separation/conversion device 100E according to a fifth embodiment of the present invention will be described with reference to FIG. 12. In addition, in the fifth embodiment, the same portions as the constituent elements in the above-described first, second, third, and fourth embodiments will be designated by the same reference signs, and a description thereof will be omitted, and only different points will be described.

FIG. 12 is a schematic view of the carbon dioxide separation/conversion device 100E according to the fifth embodiment. The carbon dioxide separation/conversion device 100E includes one or more separation units 10 which separate carbon dioxide from a carbon dioxide-containing gas to obtain a carbon dioxide-enriched gas, a reaction unit 30B which includes a catalyst for absorbing and converting the carbon dioxide in the carbon dioxide-enriched gas and causing the catalyst to absorb the carbon dioxide in the carbon dioxide-enriched gas to obtain a converted gas containing a conversion product formed of the carbon dioxide absorbed by the catalyst and hydrogen, and a hydrogen delivery unit 40 which delivers hydrogen to the reaction unit 30B. The carbon dioxide separation/conversion device 100E according to the fifth embodiment further includes a moisture removal unit 20 which removes moisture from the carbon dioxide-enriched gas, and a re-injection unit 60 which injects the converted gas again to each separation unit 10. The separation unit 10 and the moisture removal unit 20 are connected to each other through a flow path L2. The moisture removal unit 20 and the reaction unit 30B are connected to each other through a flow path L3. The reaction unit 30B and the hydrogen delivery unit 40 are connected to each other through a flow path L5. The converted gas generated in the reaction unit 30B passes through a flow path L6E and is delivered to the re-injection unit 60. The re-injection unit 60 injects the converted gas into the separation membrane module 11 of the separation unit 10 again through a flow path L10. The absorbed gas is discharged through a flow path L4. Hereinafter, each unit will be described.

The re-injection unit 60 injects the converted gas into the separation membrane module 11 of the separation unit 10 again. The re-injection unit 60 is connected to the converted gas delivery port 35B of the reaction unit 30B through the flow path L6E. The re-injection unit 60 injects the converted gas delivered through the flow path L6E into the separation membrane module 11 again through the flow path 110. Unreacted carbon dioxide in the converted gas can also be used for the carbon dioxide conversion reaction. Therefore, the yield of the conversion product can be increased.

Hereinabove, the carbon dioxide separation/conversion device 100E according to the fifth embodiment has been described above. According to the carbon dioxide separation/conversion device 100E according to the fifth embodiment, it is possible to convert carbon dioxide even when oxygen is contained because of excellent expandability of the separation membrane module 11. In addition, the absorption and conversion reaction of carbon dioxide can be simultaneously performed. In addition, the re-injection unit 60 injects the converted gas into the separation membrane module 11 again, so that the yield of the conversion product can be further improved.

Note that, the technical scope of the present invention is not limited to the above embodiment and it is possible to make various changes thereto in a range not departing from the spirit of the present invention. In addition, it is possible to appropriately replace the components in the above-described embodiments with well-known components without departing from the gist of the present invention, and the components described above may be combined as appropriate.

### Examples

Next, examples of the present invention will be described, but the conditions in the examples are one condition example adopted to confirm the implementability and the effects of the present invention, and the present invention is not limited to this one condition example. The present invention can adopt various conditions as long as the object of the present invention is achieved without departing from the gist of the present invention.

### (Production of Catalyst 1)

A catalyst was produced by a wet-type impregnation method. Al₂O₃ was obtained by sintering aluminum hydroxide at 900° C. for 3 hours. Next, an appropriate amount of an aqueous sodium nitrate solution was allowed to be impregnated into Al₂O₃ to obtain a suspension. After stirring the suspension at room temperature for 3 hours, the suspension was evaporated in a vacuum at 50° C. under reduced pressure conditions and dried at 100° C. overnight. The obtained solid was heated at 600° C. for 2 hours to obtain Na—Al₂O₃ supporting sodium. Next, in the same manner as in the case of sodium nitrate, (NH₃)₂Pt(NO₃)₂ was impregnated into Na—Al₂O₃, and the impregnated material was dried and sintered. The obtained solid was subjected to a hydrogen treatment at 350° C. to obtain Pt/Na-Al₂O₃ supporting platinum. Pt was supported in the formed of nanoparticles, and a particle diameter thereof confirmed by a scanning transmission electron microscope (STEM) was 1 to 2 nm. The amount of Pt with respect to the total mass of the catalyst was 1 wt %, and the amount of Ca was 10 wt %.

### (Production of Catalyst 2)

A catalyst was produced by a wet-type impregnation method. Al₂O₃ was obtained by sintering aluminum hydroxide at 900° C. for 3 hours. Next, an appropriate amount of an aqueous calcium nitrate solution was allowed to be impregnated into Al₂O₃ to obtain a suspension. After stirring the suspension at room temperature for 3 hours, the suspension was evaporated in a vacuum at 50° C. and dried at 100° C. overnight. The obtained solid was heated at 600° C. for 2 hours to obtain Ca—Al₂O₃ supporting calcium. Next, Ca—Al₂O₃ was impregnated into Ca—Al₂O₃ by the same method as in the case of the aqueous calcium nitrate solution, and the impregnated material was dried and sintered. The obtained solid was subjected to a hydrogen treatment at 350° C. to obtain Ni/Ca—Al₂O₃ supporting Ni, Ni was supported in the form of nanoparticles, and a particle diameter thereof confirmed by a scanning transmission electron microscope (STEM) was 1 to 2 nm. The amount of Ni with respect to the total mass of the catalyst was 10 wt %, and the amount of Ca was 30 wt %.

### (Carbon Dioxide Enrichment Properties of Separation Membrane Module)

A polydimethylsiloxane-based polymer membrane (thickness: about 150 nm) was used as a separation membrane. The separation membrane was disposed in a circular opening portion (diameter of about 5 cm) provided in a flat plate-shaped container, and the separation membrane was fixed with tape along a circumference of the opening portion to produce a separation membrane module. 10 separation membrane modules were arranged in parallel and connected to a separation membrane module connection unit. The carbon dioxide delivery port of the separation membrane module connection unit, the diaphragm pump (pressure difference formation unit), and a container for measuring the carbon dioxide concentration were connected to each other, and an inside of the separation membrane module and an inside of the separation membrane module connection unit were depressurized to measure a carbon dioxide concentration, a relative humidity, and a temperature from the start of pumping to the opening of the container for measurement. The carbon dioxide concentration, the humidity, and the temperature were disposed in a container for measurement.

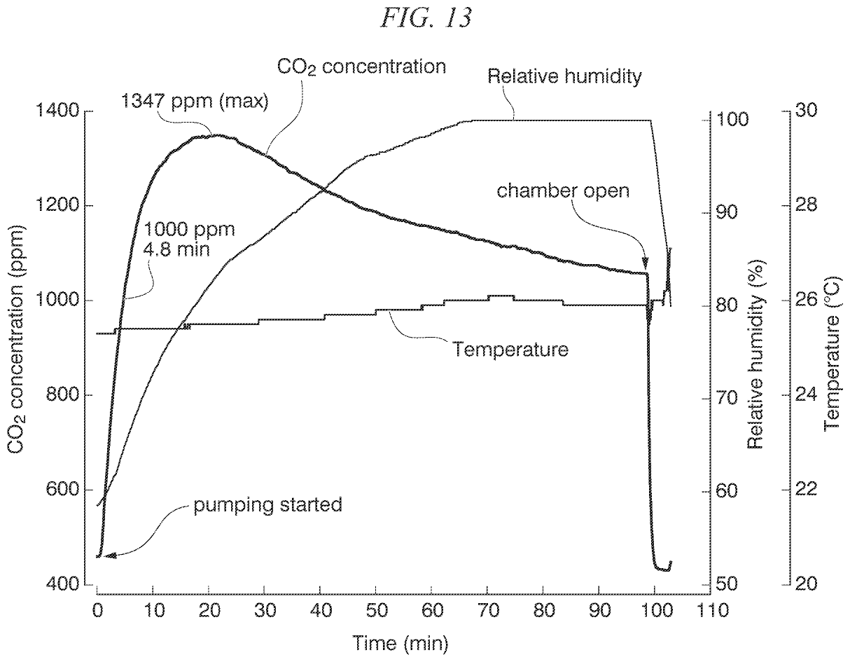

The obtained results are shown in FIG. 13. A horizontal axis indicates time (min), a first vertical axis indicates carbon dioxide concentration (ppm), a second vertical axis indicates relative humidity (%), and a third vertical axis indicates temperature (° C.). It was confirmed that the carbon dioxide concentration and the relative humidity increased over time. In addition, it was confirmed that the concentration of carbon dioxide decreased due to the enriched moisture when the relative humidity increased.

(Carbon Dioxide Enrichment Properties of Separation Membrane Module when Moisture Removal is Performed)

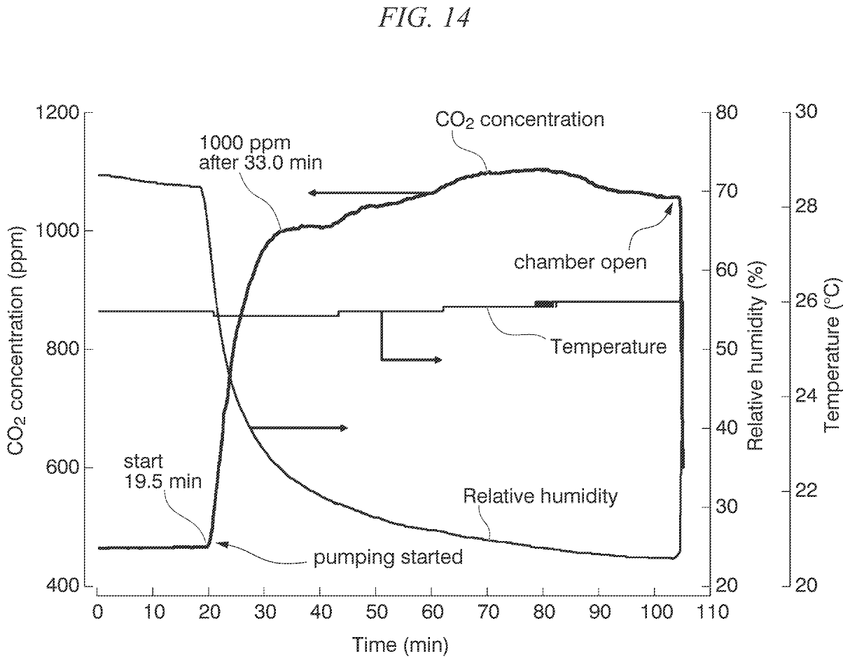

Next, in the device in which carbon dioxide enrichment properties of the separation membrane module were evaluated, a moisture trap (moisture removal unit) was connected between a measurement container and an outlet side of the diaphragm pump, and the same carbon dioxide enrichment properties were evaluated and measured. The obtained results are shown in FIG. 14. The horizontal axis indicates time (min), a first vertical axis indicates carbon dioxide concentration (ppm), a second vertical axis indicates relative humidity (%), and the third vertical axis indicates temperature (° C.). It was confirmed that the carbon dioxide concentration increased over time. In addition, since the moisture trap was present, the relative humidity did not increase, and the concentration of carbon dioxide increased stably. From the above results, it was confirmed that stable carbon dioxide enrichment can be performed because the moisture trap was provided.

### (Influence of Blowing Air on Carbon Dioxide Enrichment Properties)

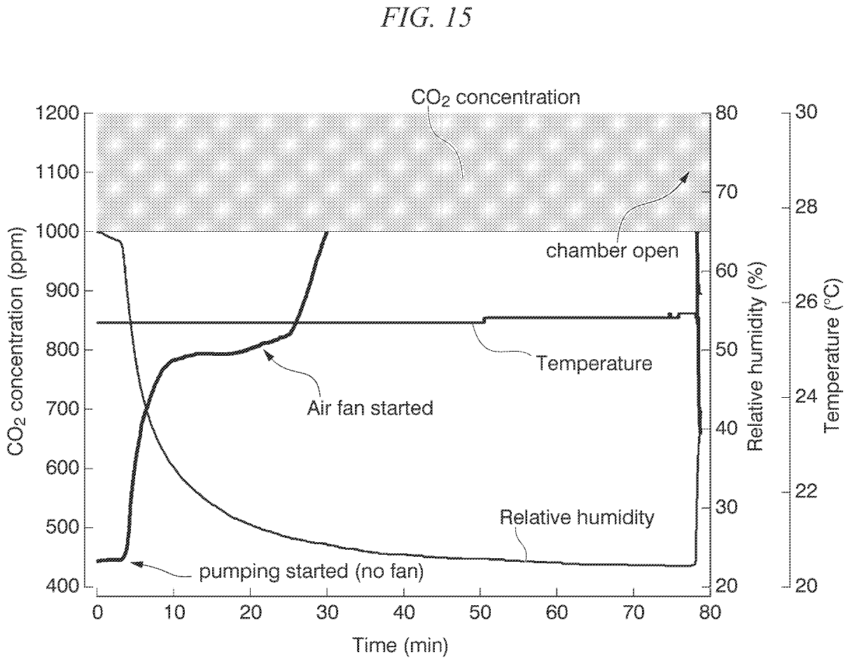

When the separation units are connected in multiple stages, it is expected that more recovery will be achieved. However, when a gas permeation amount of the separation membrane is high, supply of the gas to the separation membrane module is also important. Therefore, in the device in which the carbon dioxide enrichment properties of the separation membrane module were evaluated, the carbon dioxide enrichment properties when the gas was delivered to the separation membrane module by an air blowing system (air blowing unit) were evaluated. The obtained results are shown in FIG. 15. The horizontal axis indicates time (min), the first vertical axis indicates carbon dioxide concentration (ppm), the second vertical axis indicates relative humidity (%), and the third vertical axis indicates temperature (° C.). Until the operation of the air blowing system, the carbon dioxide concentration was around 1,000 ppm as in the above-described case, and the concentration of carbon dioxide (the carbon dioxide concentration after the separation membrane permeation) after the enrichment was slowly increased. When the air blowing system was operated, a further increase in the carbon dioxide concentration after the concentration was observed, and an enrichment effect close to about 30% was obtained.

The conversion (reduction) of carbon dioxide was evaluated using the reaction unit of FIG. 8. As reactors, two fixed bed flow reactors were used, and solids (pellet-like catalysts) having the same composition were used. The absorption of carbon dioxide into the catalyst (generation of carbonate), the flow of hydrogen to the absorbed carbon dioxide, and the generation of the conversion product were alternately performed at the same temperature and with the same type of reactor. The solids (pellet-like catalysts) having the same composition were put into two fixed-bed flow-type reactors (first reactor and second reactor) arranged in parallel as shown in FIG. 8, CO₂/air (carbon dioxide-containing gas) is allowed to flow through one reactor and hydrogen is allowed to flow through the other reactor, and a four-way valve installed upstream and downstream of the device is periodically switched, thereby continuously enabling the absorption of CO₂ (removal of O₂) and the generation of the conversion product (recovery of CO₂ absorption capacity).

The production of carbon monoxide (CO) and methane (CH₄) was performed by appropriately selecting a catalyst. For gas analysis, a reduction performance of the carbon dioxide separation/conversion device of FIG. 7 was evaluated using an infrared spectrometer and a mass spectrometer. A diaphragm pump was used for the pressure difference formation unit 14, and a cooling-type water trap and a molecular sieve were used for a cooling unit. As the separation unit, the separation membrane module evaluated in terms of carbon dioxide enrichment properties was used.

### (Methane Productivity)

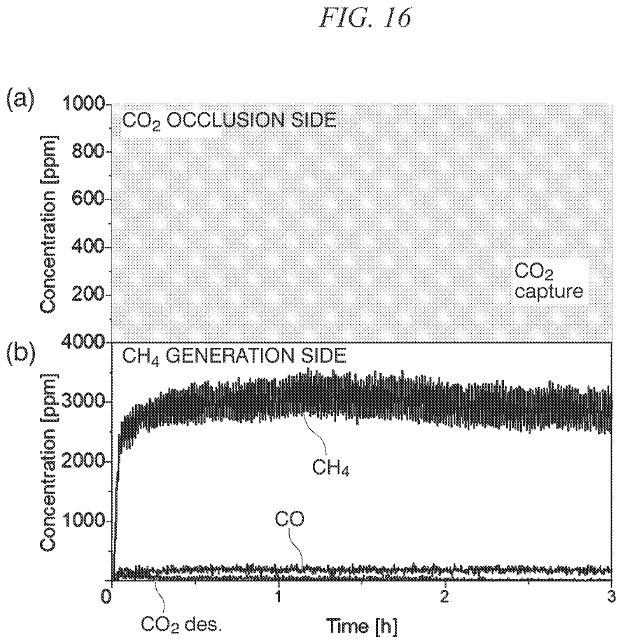

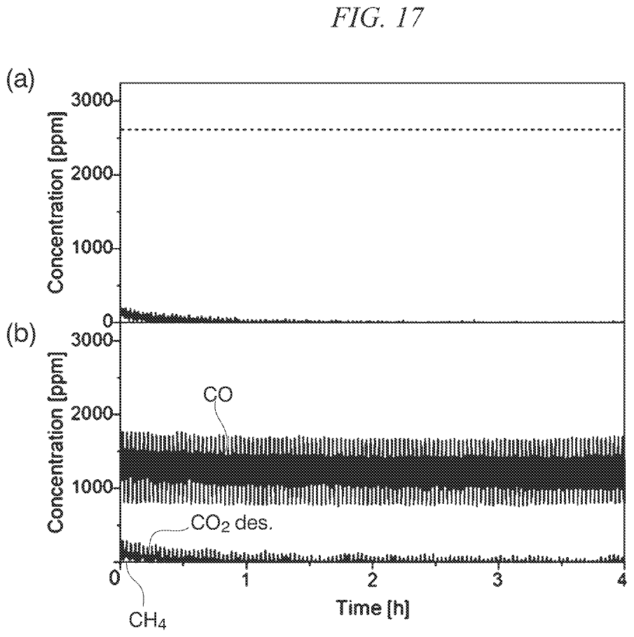

As the catalyst, a catalyst (0.5 g) in which Ni nanoparticles (10 wt %) and calcium (30 wt %) were supported on γ-alumina was used (Ni/Ca—Al₂O₃). The temperature of the reactor was 450° C. a gas switching interval of enriched CO₂/air and hydrogen was 30 seconds, and the methane productivity was evaluated. A carbon dioxide-enriched gas (concentrated CO₂/air) (flow rate of about 100 mL/min) and hydrogen (flow rate of 100 mL/min) flowed into the reaction unit to evaluate the methane productivity. The obtained results are shown in FIG. 16. (a) of FIG. 16 shows a change in gas concentration over time on the absorbed gas (CO₂ absorption) side. In (a) of FIG. 16, the horizontal axis indicates time (h), and the vertical axis indicates concentration (ppm). (b) of FIG. 16 shows results on the converted gas (methane generation) side. In (b) of FIG. 16, the horizontal axis indicates time (h), and the vertical axis indicates concentration (ppm). As shown in FIG. 16, continuous CO₂ absorption and methane production were achieved by the carbon dioxide separation/conversion device.

### (Carbon Monoxide Productivity)

As the catalyst, a catalyst (0.3 g) in which Pt nanoparticles (1 wt %) and sodium (10 wt %) were supported on γ-alumina was used (Pt/Na—Al₂O₃). The temperature of the reactor was 300° C., a gas switching interval of enriched CO₂/air and hydrogen was 60 seconds. A carbon dioxide-enriched gas (concentrated CO₂/air) (flow rate of about 100 mL/min) and hydrogen (flow rate of 100 mL/min) flowed into the reaction unit to evaluate the carbon monoxide productivity. The obtained results are shown in FIG. 17. (a) of FIG. 17 shows a change in gas concentration over time of the absorbed gas (CO₂ absorption) side. In (a) of FIG. 17, the horizontal axis indicates time (h), and the vertical axis indicates concentration (ppm). (b) of FIG. 17 shows results of the converted gas (carbon monoxide generation) side. In (b) of FIG. 17, the horizontal axis indicates time (h), and the vertical axis indicates concentration (ppm). As shown in FIG. 17, continuous CO₂ absorption and carbon monoxide production were achieved by the carbon dioxide separation/conversion device.

### INDUSTRIAL APPLICABILITY

According to the carbon dioxide separation/conversion device of the present disclosure, since carbon dioxide can be converted even if oxygen is contained because of excellent expandability of the separation membrane module, the carbon dioxide separation/conversion device has high industrial applicability.

### REFERENCE SIGNS LIST

1: Container
2: Separation membrane
10: Separation unit
11: Separation membrane module
13: Separation membrane module connection unit
14: Pressure difference formation unit
16: Carbon dioxide delivery port
20: Moisture removal unit
30: Reaction unit
31: Reactor
32: Catalyst
33: Absorbed gas discharge port
35: Converted gas delivery port
40: Hydrogen delivery unit
50: Air blowing unit
60: Re-injection unit
100: Carbon dioxide separation/conversion device

1. A carbon dioxide separation/conversion device comprising:
one or more separation units configured to separate carbon dioxide from a carbon dioxide-containing gas to obtain a carbon dioxide-enriched gas;
a reaction unit including a catalyst which absorbs and converts the carbon dioxide in the carbon dioxide-enriched gas, and configured to allow the catalyst to absorb the carbon dioxide in the carbon dioxide-enriched gas to obtain a converted gas containing a conversion product formed of the carbon dioxide absorbed by the catalyst and hydrogen; and
a hydrogen delivery unit configured to deliver the hydrogen to the reaction unit,
wherein the separation unit includes:
one or more separation membrane modules including a separation membrane which separates the carbon dioxide from the carbon dioxide-containing gas;
a separation membrane module connection unit connected to each separation membrane module and including a carbon dioxide delivery port through which the carbon dioxide-enriched gas is discharged; and
a pressure difference formation unit configured to form a pressure difference between an outside of each separation membrane module and the separation membrane module connection unit and an inside of each separation membrane module and the separation membrane module connection unit,
the separation membrane module includes:
a container including an opening portion and a discharge port through which the carbon dioxide-enriched gas is discharged to the separation membrane module connection unit; and
a separation membrane configured to cover the opening portion, is fixed along a circumference of the opening portion, and separates the carbon dioxide from the carbon dioxide-containing gas,
the reaction unit includes:
one or more reactors having the catalyst;
an absorbed gas discharge port configured to discharge an absorbed gas, which is a gas after the catalyst absorbs the carbon dioxide in the carbon dioxide-enriched gas, in each reactor;
a converted gas delivery port configured to deliver the converted gas;
a first gas switching unit connected directly or indirectly to the hydrogen delivery unit, each reactor, and the carbon dioxide delivery port; and
a second gas switching unit connected directly or indirectly to the absorbed gas discharge port, each reactor, and the converted gas delivery port,
when the catalyst absorbs the carbon dioxide, the first gas switching unit connects at least one of the reactors to the carbon dioxide delivery port, and the second gas switching unit connects the reactor connected to the carbon dioxide delivery port to the absorbed gas discharge port, and
when the carbon dioxide absorbed by the catalyst reacts with the hydrogen, the first gas switching unit connects at least one of the reactors to the hydrogen delivery unit, and the second gas switching unit connects the reactor connected to the hydrogen delivery unit to the converted gas delivery port.

2. The carbon dioxide separation/conversion device according to claim 1,
wherein the reaction unit includes a first reactor and a second reactor,
when the first gas switching unit connects the first reactor to the carbon dioxide delivery port, the first gas switching unit connects the second reactor to the hydrogen delivery unit in a state where the second reactor and the carbon dioxide delivery port are not connected to each other, and the second gas switching unit connects the first reactor to the absorbed gas discharge port, and connects the second reactor to the converted gas delivery port in a state where the second reactor and the absorbed gas discharge port are not connected to each other, and
when the first gas switching unit connects the second reactor to the carbon dioxide delivery port, the first gas switching unit connects the first reactor to the hydrogen delivery unit in a state where the first reactor and the carbon dioxide delivery port are not connected to each other, and the second gas switching unit connects the second reactor to the absorbed gas discharge port, and connects the first reactor to the converted gas delivery port in a state where the first reactor and the absorbed gas discharge port are not connected to each other.

3. The carbon dioxide separation/conversion device according to claim 1,
wherein the reaction unit includes a first reactor,
when the catalyst absorbs the carbon dioxide, the first gas switching unit connects the first reactor to the carbon dioxide delivery port in a state of not being connected to the hydrogen delivery unit, and the second gas switching unit connects the first reactor to the absorbed gas discharge port in a state of not being connected to the converted gas delivery port, and
when the carbon dioxide absorbed by the catalyst reacts with the hydrogen, the first gas switching unit connects the first reactor to the hydrogen delivery unit in a state of not being connected to the carbon dioxide delivery port, and the second gas switching unit connects the first reactor to the converted gas delivery port in a state of not being connected to the absorbed gas discharge port.

4. The carbon dioxide separation/conversion device according to claim 1,
wherein the carbon dioxide-enriched gas discharged from the first separation unit among the separation units is injected into the second separation unit among the separation units.

5. The carbon dioxide separation/conversion device according to claim 1,
wherein the separation membrane contains a polydimethylsiloxane-based material as a main component.

6. The carbon dioxide separation/conversion device according to claim 1, further comprising:
an air blowing unit configured to deliver the carbon dioxide-containing gas to each separation membrane module.

7. The carbon dioxide separation/conversion device according to claim 1, further comprising:
a moisture removal unit configured to remove moisture in the carbon dioxide-enriched gas.

8. The carbon dioxide separation/conversion device according to claim 1, further comprising:
a re-injection unit configured to inject the converted gas into each separation membrane module again.

9. The carbon dioxide separation/conversion device according to claim 1,
wherein the conversion product is carbon monoxide.

10. The carbon dioxide separation/conversion device according to claim 1,
wherein the conversion product is methane.
